(19) United States
(12) Patent Application Publication
(10) Pub. No.: US 2018/0140225 A1
(43) Pub. Date: May 24, 2018

(54) BODY PART DEFORMATION ANALYSIS USING WEARABLE BODY SENSORS

(71) Applicant: Figur8, Inc., Boston, MA (US)

(72) Inventors: Nan-Wei Gong, Cambridge, MA (US); Jennifer Maria Brine, Somerville, MA (US); Tiegeng Ren, Westford, MA (US)

(73) Assignee: Figur8, Inc., Boston, MA (US)

(21) Appl. No.: 15/875,311

(22) Filed: Jan. 19, 2018

Related U.S. Application Data
(63) Continuation-in-part of application No. 15/271,863, filed on Sep. 21, 2016.
(60) Provisional application No. 62/448,525, filed on Jan. 20, 2017.
(60) Provisional application No. 62/464,443, filed on Feb. 28, 2017.
(60) Provisional application No. 62/513,746, filed on Jun. 1, 2017.
(60) Provisional application No. 62/221,590, filed on Sep. 21, 2015.

(51) Int. Cl. A61B 5/107; A61B 5/00; A61B 5/11

(52) U.S. Cl. CPC A61B 5/1071; A61B 5/1072; A61B 5/6804; G01N 27/221; A61B 5/7425; A61B 5/1126; A61B 5/1123; A61B 5/1121

(57) ABSTRACT

Disclosed embodiments describe techniques for body part analysis. Data is collected from a body sensor. The body sensor is coupled to a fabric attached to a body part. The body sensor provides electrical information based on a deformation of the body part. The data from the body sensor is analyzed to determine the deformation of the body part. An angle for the body part is determined based on the deformation. The data is augmented with additional data collected from an inertial measurement unit. Anchor points for the body part are determined, where the anchor points enable placement of the body sensor coupled to the fabric. The body part is treated, wherein the body part treatment is based on the analyzing. Additional data is collected from a second body sensor. The second body sensor is also coupled to the fabric.

### RELATED APPLICATIONS

This application claims the benefit of U.S. provisional patent applications “Body Part Deformation Analysis with Wearable Body Sensors” Ser. No. 62/448,525, filed Jan. 20, 2017, “Body Part Deformation Analysis using Wearable Body Sensors” Ser. No. 62/464,443, filed Feb. 28, 2017, and “Body Part Motion Analysis with Wearable Sensors” Ser. No. 62/513,746, filed Jun. 1, 2017.

This application is also a continuation-in-part of U.S. patent application “Electronic Fabric for Shape Measurement” Ser. No. 15/271,863, filed Sep. 21, 2016, which claims the benefit of U.S. provisional patent application “Electronic Fabric for Shape Measurement” Ser. No. 62/221,590, filed Sep. 21, 2015.

Each of the foregoing applications is hereby incorporated by reference in its entirety.

### FIELD OF ART

This application relates generally to body part analysis and more particularly to body part deformation analysis using wearable body sensors.

### BACKGROUND

The accurate measurement of a given shape has many applications in the fields of machine vision, industrial automation, scientific research, and recycling/reclamation, among others. The shapes that are measured include objects of interest, manufactured parts, etc. Shape measurements are used for object differentiation, where the object differentiation is based on material, size, shape, and cost, among other parameters. When the shape being measured is a portion of a human body, then shape measurement has further applications in industries such as fashion and healthcare. While the former is used to determine the proper fit of clothing, accessories, and equipment, the latter is used to obtain data related to personal medical information and to design medical treatments. Proper medical treatments are essential for comfort, safety, and therapeutic outcomes.

In a clinical setting, accurate and precise human body measurements are difficult to obtain. For example, consider a relatively simple, static, volumetric body part measurement, such as measuring the volume of fluid buildup in a limb caused by lymphedema. This is typically a manual process where a tape measure is used by a clinical professional to take body measurements. First the limb is marked along a longitudinal axis using the tape measure and a marking pen. An appropriate gradation, every 1 cm for example, is marked. Next, a transverse circumference is measured at every gradation and recorded. The transverse circumferential measurements are repeated along the desired length of the limb. At a subsequent clinical visit, perhaps one week or one month later, the measurements are taken again. Total limb volume V can be approximated by assuming a step-wise linear series of cylindrical disks. The volume V can be expressed as the area A of each transverse cross-section (where A=C²/4π, and where C is the measured circumference) times the height h of each gradation, and then the sum of all the cylindrical disk volumes is determined as the total volume. In this way, lymphedema progression and/or treatment effectiveness can be monitored.

Unfortunately, even though this is a relatively simple example involving a static measurement of a stationary body part, the typical clinical approach is fraught with inconsistencies and opportunities for human error. A different person may be taking the measurements. Inconsistent pressure may be applied when measuring the circumference. The tip of the marking pen can be several mm wide. Subtle limb shape changes, whether related to lymphedema or not, may greatly affect the accuracy of the estimated volumetric model calculation.

While taking static body part measurements is very difficult, it is even more difficult to measure moving body parts, such as a joint. Body part joint movement is three-dimensional, and the movement happens in real-time, that is, it is non-static. By necessity, the body part joint is moving when a measurement needs to be taken. Body part joint measurements can involve different deformations along multiple axes. Multiple measurements of a repetitive motion may be required. Measurements may need to be made while the body part is under a load condition or under nominal conditions. All of these variables present additional layers of variation that makes measurement difficult. Accordingly, a great need exists to be able to accurately measure and analyze body part deformation.

### SUMMARY

The proper determination of the deformation of a body part is critical to measuring the capabilities and parameters of the body part and to designing therapies for the body part. Techniques are disclosed for body part deformation analysis with wearable body sensors. The wearable body sensors can be coupled to a fabric which can be attached to a body part. The body part can include one or more of a knee, shoulder, elbow, wrist, hand, finger, thumb, ankle, foot, toe, hip, torso, spine, arm, leg, neck, jaw, head, or back. The body part can be a joint, and the deformation can include the bending of said joint. The fabric can be a tape, a garment, and so on. The tape can be a specialized tape such as a physical therapy tape, surgical tape, therapeutic kinesiology tape, and so on. The garment can include a cuff, a strap or a belt, or an article of clothing such as socks, pants, shirts, hats, gloves, etc. The body sensors that are coupled to the fabric provide data including electrical information. The electrical information can include capacitance, resistance, impedance, inductance, and so on. The data from the body sensor can be analyzed to determine a deformation of the body part. The deformation of the body part can then be used to determine a variety of parameters related to the body part such as flexion, rotation, and so on. The electrical information can provide information on an angle through which a joint bends. The analysis of the electrical information can be used to identify an abnormality and/or to propose a body part treatment. The body part treatment can include one or more of medical, physical therapy, occupational therapy, athletic training, strengthening, flexibility, endurance, conditioning, habilitation therapy, or rehabilitation therapy treatment.

A second body sensor can also be coupled to the fabric. The second body sensor can be coupled to a second fabric portion. The second body sensor can provide information on deformation of a joint beyond an angle of deformation, such as the rotation of a joint. In embodiments, data collected includes rotation of the joint, flex parameters of the joint, and varus and valgus tendencies of the joint. In embodiments, additional data is collected from a second body sensor, wherein the additional data from the second body sensor provides information on deformation of the joint beyond an angle of deformation for the joint and rotation of the joint. A processor-implemented method for body part analysis is disclosed comprising: collecting data from a body sensor, wherein the body sensor is coupled to a fabric attached to a body part and the body sensor provides electrical information based on a deformation of the body part; and analyzing the data from the body sensor to determine the deformation of the body part. An apparatus for body part analysis is disclosed comprising: a body sensor coupled to a fabric, wherein the fabric is attachable to a body part and wherein the body sensor provides electrical information based on a deformation of the body part; and a processor coupled to the body sensor, wherein the processor analyzes the electrical information from the body sensor to determine the deformation of the body part.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for sensor analysis of a body part.

FIG. 2 is a flow diagram for body part deformation analysis.

FIG. 3 is a block diagram for body part deformation analysis.

FIG. 4 shows fabric strips with sensors.

FIG. 5 shows an example analysis of deformation electrical information.

FIG. 6 illustrates detail of a capacitive sensor.

FIG. 7 illustrates fabric/sensor detail.

FIG. 8 illustrates fabric/sensor detail according to a first implementation.

FIG. 9 illustrates fabric/sensor detail according to a second implementation.

FIG. 10 illustrates fabric/sensor detail according to a third implementation.

FIG. 11 shows anchor points for sensor placement on a leg.

FIG. 12 illustrates anchor point anatomical detail.

FIG. 13 shows right knee sensor attachment.

FIG. 14 illustrates electrical information from sensors.

FIG. 15 provides steps for knee evaluation testing.

FIG. 16 illustrates deformation analysis.

FIG. 17 illustrates deformation analysis of symmetric body parts.

FIG. 18 is a flow diagram for body part sensor assembly.

FIG. 19 is a system for body part analysis.

### DETAILED DESCRIPTION

Techniques are disclosed for body part deformation analysis with wearable body sensors. The wearable body sensors can be coupled to a fabric which can be attached to one or more body parts. The body sensors can be used to measure various parameters relating to the body parts, to compare body parts, and so on. The measurement data of one or more body parts can be used to assist in a diagnosis, plan a therapy, measure progress of a therapy, and so on. The body part can include one or more of a knee, shoulder, elbow, wrist, hand, finger, thumb, ankle, foot, toe, hip, torso, spine, arm, leg, neck, jaw, head, or back. The fabric can be a tape, a garment, or other fabric that can be attached to a body part. The fabric can be integrated in or can comprise a brace for a body part, such as a knee brace. The tape can be a specialized tape such as a physical therapy tape, surgical tape, therapeutic kinesiology tape, and so on. The concepts include using body tape for attachment to anchor joints, muscles, and specific bone structures for motion capture. Because the body tape attaches directly to the body, this tape usage and motion capture is superior with greater precision than other techniques including image capture/vision analysis.

The garment can include a cuff, a strap, a belt, and/or an article of clothing such as socks, pants, shorts, shirts, hats, and gloves, etc. The body sensors that are coupled to the fabric provide data including electrical information. The electrical information can include capacitance, resistance, impedance, inductance, and so on. The electrical information can include changes in capacitance, resistance, impedance, inductance, etc. The data from the body sensor can be analyzed to determine a deformation of the body part. The deformation of the body part can be used to determine a variety of parameters related to the body part such as flex, rotation, and so on. The analysis of the electrical information can be used to assist in a medical diagnosis. The analysis of the electrical information can be used to propose a treatment for the body part. The body part treatment can include one or more of medical, physical therapy, occupational therapy, athletic training, strengthening, flexibility, endurance, conditioning, habilitation therapy, or rehabilitation therapy treatment. Paired body parts, such as left and right arms or shoulders or legs can be analyzed to show subtle differences in deformation capabilities.

The garment can comprise non-stretchable fabric. The garment that comprises non-stretchable fabric can lack non-stretchable fabric where the sensor module resides. In embodiments, the garment that comprises non-stretchable fabric has no fabric where the sensor module resides. In embodiments, the garment that comprises non-stretchable fabric can further comprise stretchable fabric where the sensor module resides. The garment can comprise fabric that is substantially stretchable in only one direction, which can be called the primary stretchable direction. When a direction of stretch in a fabric is less than 10% stretchable when compared to the primary stretchable direction, it can be considered substantially stretchable in only one direction, given that the direction of stretch is at least at a 20° angle from the primary direction. In embodiments, the entirety of the garment which encompasses the body portion can be stretchable. In embodiments, the sensor module can be integrated within the garment that encompassing the body portion. The sensor of the sensor module can be printed on the fabric with the sensor module electronics connectable thereto. The sensor of the sensor module can be laminated in the fabric with the sensor module electronics either included in the lamination structure or connectable thereto. The sensor module can be sewn onto the garment or hooked onto the garment. In embodiments, the amount that the sensor module is stretched can measure a torso diameter, a torso length, a neck diameter, an arm diameter, an arm length, a leg diameter, a leg length, a foot diameter, or a foot length. In embodiments, the sensor and/or the sensor module are resistant to water and therefore washable. A washable sensor and/or sensor module provides for easy reuse, sanitization, and the like.

The assembly of a fabric with integrated deformation sensor is provided. The assembly can comprise various combinations of adhesive, base material, sensors, electrical connections/interconnections/couplings, and electronics. In a certain assembly, one layer of adhesive is applied. In another certain assembly, two layers of adhesive are applied. In yet another assembly, three layers of adhesive are applied. The assembly may include sensors for determining deformation-based changes in the electrical properties of capacitance, resistance, impedance, or inductance, or a combination of the electrical properties. The assembly may include connective conductors for coupling the sensor which can be discrete wires, printed wires, low resistance wires, high resistance wires, optical waveguides, and so on. The assembly may integrate electronics coupled to the conductors to facilitate providing deformation electrical information.

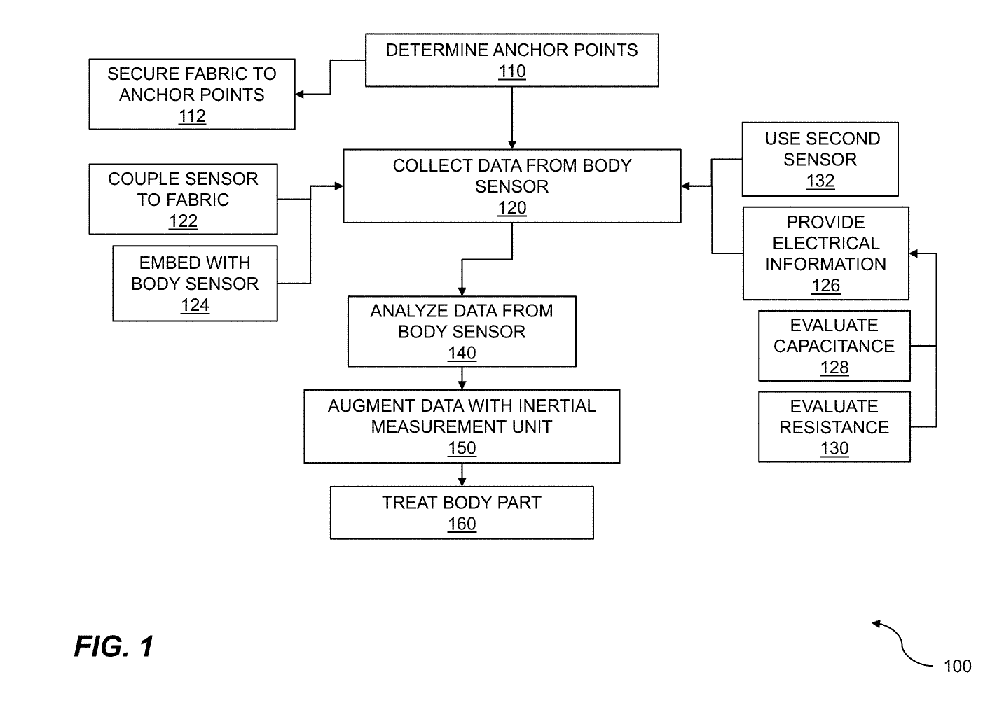

FIG. 1 is a flow for body part analysis. Analysis of body part deformation uses wearable body sensors. Data is collected from a body sensor. The body sensor is coupled to a fabric attached to a body part. In embodiments, the fabric attached to the body part can include tape, where the tape can be physical therapy tape, therapeutic kinesiology tape, and so on. In other embodiments, the fabric attached to the body part can include a garment, a brace, and so on. The fabric can include an adhesive, a belt, a strap, and so on, for attaching to the body part. The body part can include a joint or articulation in the body. The deformation can include bending of the joint. The body sensor provides electrical information based on a deformation of the body part. The electrical information can provide information on an angle through which the joint bends, or articulates. The electrical information can include capacitance, resistance, and so on. The data from the body sensor is analyzed to determine the deformation of the body part. The deformation of the body part can be used for measurement and treatment purposes. The flow 100 includes determining anchor points 110 for the body part, where the anchor points can enable placement of the body sensor coupled to the fabric 122. The anchor points, as described elsewhere, can be used to identify, monitor, measure, treat, etc. the body part to which a sensor can be attached. The anchor points can be related to a particular body part, such as two or more anchor points for collecting data on one or more cartilages in a knee. Multiple anchor points can be associated with a given body part. The body part can be one or more of a knee, shoulder, elbow, wrist, hand, finger, thumb, ankle, foot, toe, hip, torso, spine, arm, leg, neck, jaw, head, or back. The flow 100 can include securing the fabric to the anchor points 112.

The flow 100 includes collecting data from a body sensor 120. Some embodiments can use a second sensor 132, and therefore the body sensor can comprise two or more body sensors. The collecting can comprise collecting data from a second body sensor. The second body sensor can be coupled to a second fabric portion. The body sensor can be coupled to a fabric 122 attached to a body part. The fabric can be a knitted fabric, a woven fabric, an embroidered fabric, a Jacquard weave fabric, and so on. In embodiments, the woven material comprises a woven body sensor. The fabric attached to the body part can include tape. The tape can be any of a variety of tapes including adhesive tape, waterproof tape, paper tape, surgical tape, pressure sensitive tape, double-sided tape, and so on. In embodiments, the tape comprises physical therapy tape. In other embodiments, the tape comprises therapeutic kinesiology tape. The fabric can include a garment, where the garment can include a hat, a shirt, pants, a cuff, socks, or any other garment that can be applied to a body part. Other sensors can be used for collecting additional data from a body sensor. In embodiments, the data from the body sensor can be augmented with additional data collected from an inertial measurement unit 150. The inertial measurement unit can be used to measure linear and angular motion. In embodiments, the inertial measurement unit can be embedded with the body sensor 124. The body sensor provides electrical information 126 based on a deformation of the body part. Various types of electrical information can be provided by the body sensor. The body sensor can evaluate a variety of electrical signals. In embodiments, the body sensor evaluates capacitance 128 and/or resistance 130. In further embodiments, the body sensor evaluates impedance, inductance, reluctance, conductance, reactance, and so on. In further embodiments, the body sensor evaluates a combination of electrical characteristics, such as resistance and capacitance, to name just one possible combination.

The flow 100 includes analyzing the data from the body sensor 140 to determine the deformation of the body part. The analysis and determination of deformation can be performed for a variety of purposes such as diagnostic, metric, therapeutic, and so on. In embodiments, the analyzing can include performing a symmetry evaluation. The symmetry evaluation can be used to determine range of motion, flexibility, and so on. The symmetry evaluation can include an evaluation of a similar body part. A right knee can be compared to a left knee, a right hip to a left hip, and so on. An injured knee can be compared to a healthy knee, a damaged wrist can be compared to a repaired wrist, and so on. The symmetry evaluation can include an evaluation of a symmetrical operation for the body part. The symmetry evaluation can include range of motion forward and backward, range of flex, etc. Other analysis techniques can be applied. In embodiments, the analyzing can include evaluation of bone structure, muscle structure, ligament structure, or body part motion.

Returning to the analyzing for determination of deformation of the body part, the deformation of the body part can be used for additional purposes. In embodiments, deformation includes a displacement. The displacement can be forward and backward, side to side, up and down, and so on. In other embodiments, the deformation can include an angular motion. The angular motion can include a rotation, a flex, and so on. The deformation can be used to determine location, angle, displacement, and so on. In embodiments, the determining an angle for the body part can be based on the deformation. The determining the deformation can be used for medical and therapeutic purposes. In embodiments, the analyzing can be performed to compare preoperative deformation with postoperative deformation. Success of a surgery can be gauged by measured improvement in joint rotation, flexion, and so on. In other embodiments, the analyzing can be performed to compare deformation over time as part of physical therapy. The therapy, such as physical therapy, can be measured and adapted as necessary to improve an outcome of the therapy. The analysis of the deformation can include an anatomical evaluation. The anatomical evaluation can be used to determine the need for a surgery, the appropriateness of a surgery, etc. The anatomical evaluation can determine a variety of characteristics related to a given body part. The deformation can comprise a range of motion of the body part such as whether or not the range of motion of the body part is restricted. The deformation can comprise a ligament laxity, where the degree of laxity can be determined. The deformation can include the stability of the body part. The stability of the body part can be significant to personal safety, pain severity, and so on. The stability can be analyzed over multiple repetitions to determine whether the body part moves in the same manner throughout the repetitions. Physiological parameters can be monitored during repetitive body part motions. The physiological parameters can include respiration, heart beats, heart beat variability, muscle twitching, and so on.

The flow 100 includes augmenting the data with additional data collected from an inertial measurement unit 150. The inertial measurement unit (IMU) can measure linear motion, angular motion, etc. The IMU can include gyroscopes, accelerometers, and so on. The IMU can provide angular velocity, acceleration, and so on. One or more inertial measurement units can provide the additional data. The one or more inertial measurement units can be embedded with the body sensor coupled to a fabric. The data and the additional data can be used in a virtual reality (VR) representation of the body. Virtual reality based body representations can include a realistic, simulated view of the movement or structure of body parts. Virtual reality can include augmented reality, where actual body part structure and movement is combined with body part sensor deformation and inertial measurement data to provide a composite body part representation. For example, the data and the additional data collected by one or more body sensors and one or more inertial measurement units, while coupled to an injured knee joint, can be assimilated into a VR view of the joint. The VR view is then suitable for diagnostic, pedagogical, or treatment purposes. The data and the additional data can be used for three-dimensional (3D) joint analysis. The 3D joint analysis can be combined with a VR representation to provide a 3D skeleton reconstruction. In embodiments, only one joint angle is required to provide the skeleton reconstruction.

Many entertainment media, such as computer animated movies and video games involve simulated human body part movement. Natural body part deformation and joint movement is an integral part of making such computer-generated characters appear realistic. The simple movement of a human being walking is actually an extremely complex series of multiple body part deformations and joint movements. In embodiments, 3D tracking, joint movement and/or skeleton reconstruction virtual reality components can be provided for an entertainment system. In embodiments, the body parts comprise animal or other non-human body parts.

The flow 100 includes body part treatment 160, wherein the body part treatment is based on the analyzing. Various types of body part treatment can be proposed. Results of the body part treatment can be gauged. In embodiments, the body part treatment comprises one or more of medical, physical therapy, occupational therapy, athletic training, strengthening, flexibility, endurance, conditioning, or rehabilitation therapy treatment. Various steps in the flow 100 may be changed in order, repeated, omitted, or the like without departing from the disclosed concepts. Various embodiments of the flow 100 can be included in a computer program product embodied in a non-transitory computer readable medium that includes code executable by one or more processors.

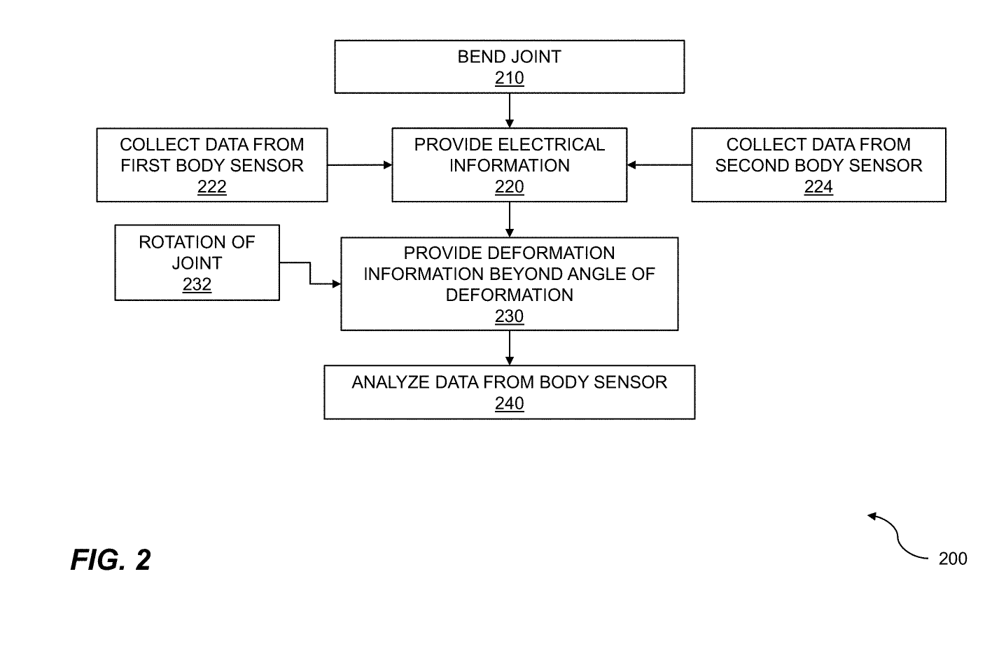

FIG. 2 is a flow diagram for body part deformation analysis. Body part deformation analysis is based on wearable body sensors that are coupled to a fabric attached to a body part. The body part is bent at a joint to produce the deformation. Data is collected from one or more body sensors. The data collected from the one or more body sensor includes electrical information based on the deformation of the body part joint. The data can include information regarding the angle of deformation of the joint of the body part. The data can include information beyond the angle of deformation, including rotation of the joint, flexion parameters of the joint, varus and valgus tendencies of the joint, and so on. A second body sensor can provide information on deformation of a joint beyond an angle of deformation for the joint. A second body sensor can provide information on rotation of the joint

The flow 200 includes collecting data from a first body sensor 222. The data collected from the first body sensor 222 provides electrical information 220. The provided electrical information 220 can include deformation information about the bending of a joint 210. The deformation information can include the angle of the deformation of the joint. The angle of deformation can be provided directly from the sensor or derived from analysis of the data. The flow 200 includes collecting data from a second body sensor 224. The collected data from the second body sensor can also provide electrical information 220. The electrical information provided for both the first body sensor and the second body sensor can be for the deformation of the same joint. More than two body sensors can be used to collect additional data and to provide electrical information 220 for the same joint. Embodiments can include providing deformation information beyond the angle of deformation 230. The deformation information beyond the angle of deformation can be enabled by using two or more body sensors on the same joint. The provided deformation information beyond the angle of deformation 230 can include the rotation of the joint 232. Providing the electrical information 220 facilitates analyzing data from a body sensor 240.

Other information beyond the angle of deformation can be provided by the body sensors. For example, a human right knee joint can have a first body sensor attached to its outside, rightmost side surface. A second body sensor can be attached to the right knee inside, leftmost side surface. A right knee joint with no varus or valgus tendencies may display similar left- and right-side deformation information throughout the bending of the joint. However, a right knee joint with varus tendencies may display a greater deformation on the outside sensor, thus indicating a degree of being bowlegged. Similarly, a right knee joint with valgus tendencies may display a greater deformation on the inside sensor, thus indicating a degree of being knock-kneed. Many other such abnormalities can be diagnosed using one or more body sensors.

Another example involves collecting data from one or more body sensors on each of two symmetric body parts. For example, a right knee and a left knee can each have two or more body sensors collecting data from the symmetric joints. Other such joints can include hands, wrists, elbows, shoulders, hips, ankles, and feet, etc. The analyzing the data from the body sensors 240 can include symmetry evaluations, such as differences in range of motion between symmetric body parts and joints.

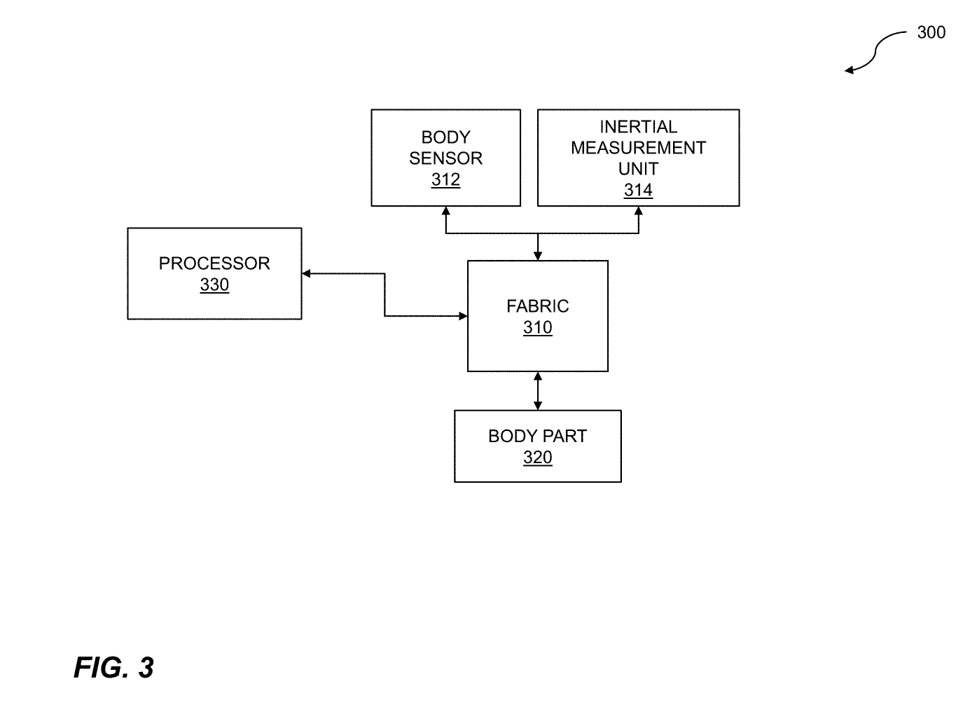

FIG. 3 is a block diagram for body part deformation analysis. Body part deformation analysis is based on wearable body sensors that are coupled to a fabric attached to a body part. Data is collected from a body sensor. The body sensor provides electrical information based on a deformation of the body part. The data from the body sensor, including electrical information, is analyzed to determine the deformation of the body part. Body part treatment can be based on the analysis of the level of deformation of the body part. Body part treatment can include one or more of medical techniques, physical therapy, occupational therapy, athletic training, strengthening, flexibility, endurance, conditioning, or rehabilitation therapy treatment.

A block diagram for body part deformation analysis 300 is shown. The block diagram 300 includes fabric 310. The fabric 310 can include a tape, a woven material, etc. The fabric 310 can be attached to a body part 320, where the body part 320 can include one or more of a knee, shoulder, elbow, wrist, hand, finger, thumb, ankle, foot, toe, hip, torso, spine, arm, leg, neck, jaw, head, back, and so on. The fabric 310 can be coupled to a body sensor 312. The body sensor 312 can include two or more body sensors. The body sensor can collect electrical information including capacitance, resistance, impedance, inductance, and so on. The body sensor 312 can include integrated electronics that aid in the capture of the electrical information. The integrated electronics can include circuitry for converting a deformation-based change in an electrical characteristic of the body sensor 312 into an electrical signal. The electrical signal can be analog-based or digital-based. The fabric 310 can be coupled to an inertial measurement unit (IMU) 314. The IMU 314 can comprise multiple IMUs. The IMU 314 can be embedded in the fabric 310. The IMU can capture movement information, attitude information, position information, acceleration information, etc. The fabric 310 can be coupled to a processor 330. The processor 330 can be used for controlling the one or more body sensors, for collecting data from the body sensors, for analyzing data from the body sensors, and so on.

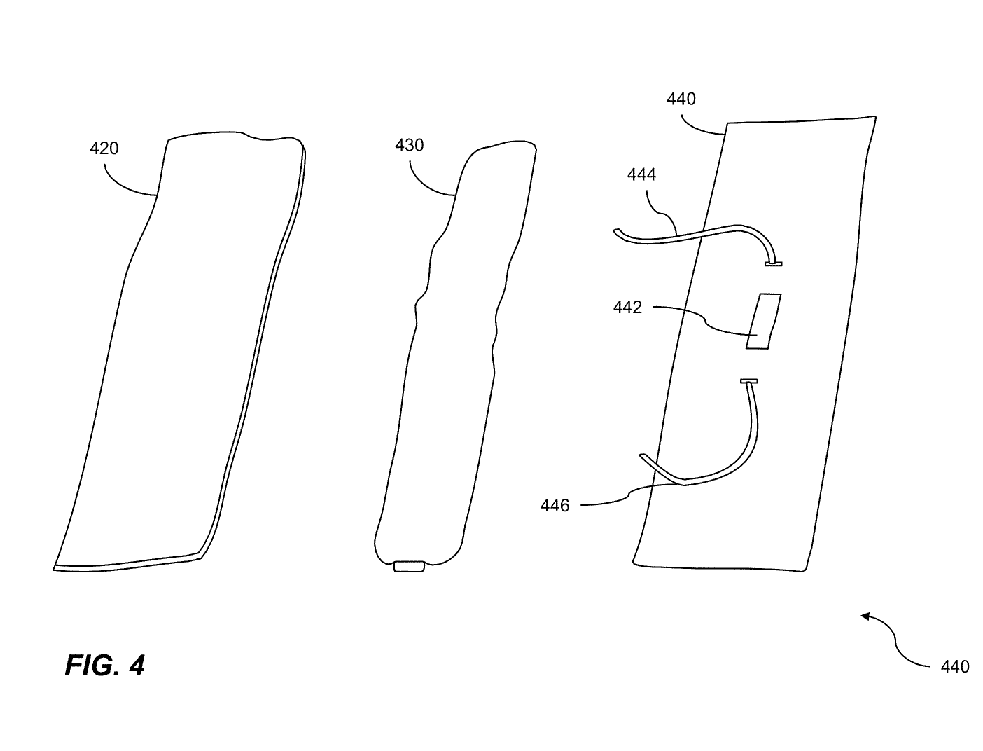

FIG. 4 shows fabric strips with sensors 400. Wearable body sensors are used to analyze body part deformation. The wearable body sensors can include an embroidered body sensor, a woven body sensor, and so on. In embodiments, the body sensor is stitched to or otherwise bonded with the fabric strip or tape. The wearable body sensors can include tape such as physical therapy tape, therapeutic kinesiology tape, etc. The body sensor can be applied to a body portion, worn on the body portion, encompass a body portion, etc. The wearable sensors can be used for collecting data from the body portion, where the data can be electrical information. The electrical information can include capacitance, resistance, impedance, inductance, etc. The body sensor can be coupled to a fabric attached to a body portion, and the body sensor can provide electrical information such as capacitance, resistance, impedance, etc. The fabric can include a garment such as a sock, hat, shirt, pants, belt, and so on. The electrical information can be based on a deformation of the body part. Data collected from the body sensor can be analyzed to determine the deformation of the body part. The data relating to the deformation of the body part can be used for body part treatment including medical techniques, physical therapy, occupational therapy, athletic training, strengthening, flexibility, endurance, conditioning, or rehabilitation therapy treatment. Three fabric strips 420, 430, and 440 are shown in the FIG. 4. Various fabric garments (not shown) can also be used for collecting data relating to a body part. The three fabric strips 420, 430, and 440 can include one or more body sensors. An illustrative body sensor 442 is shown. The sensors can be coupled to the fabric strips, where the fabric strips can include a woven material. In embodiments, the woven material comprises an embroidered body sensor. In other embodiments, the woven material comprises a woven body sensor.

The sensor 442 can include coupling wires 444 and 446 for electrical connection from the sensor to separate electronics. The sensor can change electrical properties as it undergoes deformation which results from being attached to a body part or body part joint that flexes. Coupling wires 444 and 446 facilitate communication of the changes in electrical properties. The coupling wires can be in the form of discrete wires, or they can be in any other form that facilitates data transmission such as printed wires, woven strand wires, optical wave guides, electromagnetic signaling channels, and so on.

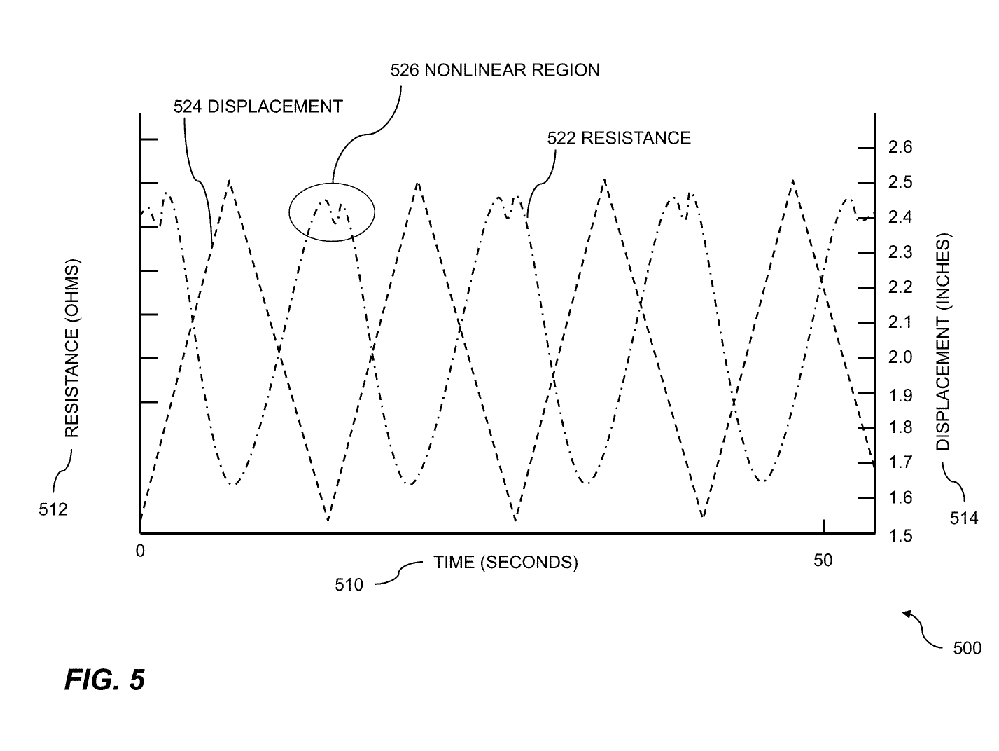

FIG. 5 shows an example analysis of deformation electrical information. The example 500 is in the form of a graph in which the horizontal axis represents time 510. The left-hand vertical axis shows resistance 512, while the right-hand vertical axis shows displacement 514. The complex dashed line (dashes and dots) shows the change of resistance 522 over time. The change of resistance 522 is derived from the change of electrical properties of a sensor as it is deformed. The value of the changing resistance is noted on the left-hand resistance 512 axis. The deformation is shown by the simple dashed line (dashes only) and represented by the displacement 524 graph line. The value of the derived displacement is noted on the right-hand displacement 514 axis. As can be observed from example 500, the resistance 522 and displacement 524 are inversely related. In other words, for this particular sensor example, the sensor's resistance decreases as it is stretched, and vice-versa. Other relationships between sensor electrical property changes and deformation can be included.

The example 500 shows a non-linear region 526. The non-linear region 526 can be a result of the sensor reaching the end of its intended linear range. For example, stretching the sensor beyond 2.4 inches, as shown on displacement axis 514, may produce a complex resistance result that cannot be understood through a simple linear relationship. Analyzing the resistance can include estimating the displacement when such a non-linear region 526 occurs. Alternatively, integrated electronics can be used to compensate for operation in a non-linear region. In some embodiments, only linear relationships exist between deformation and electrical characteristic.

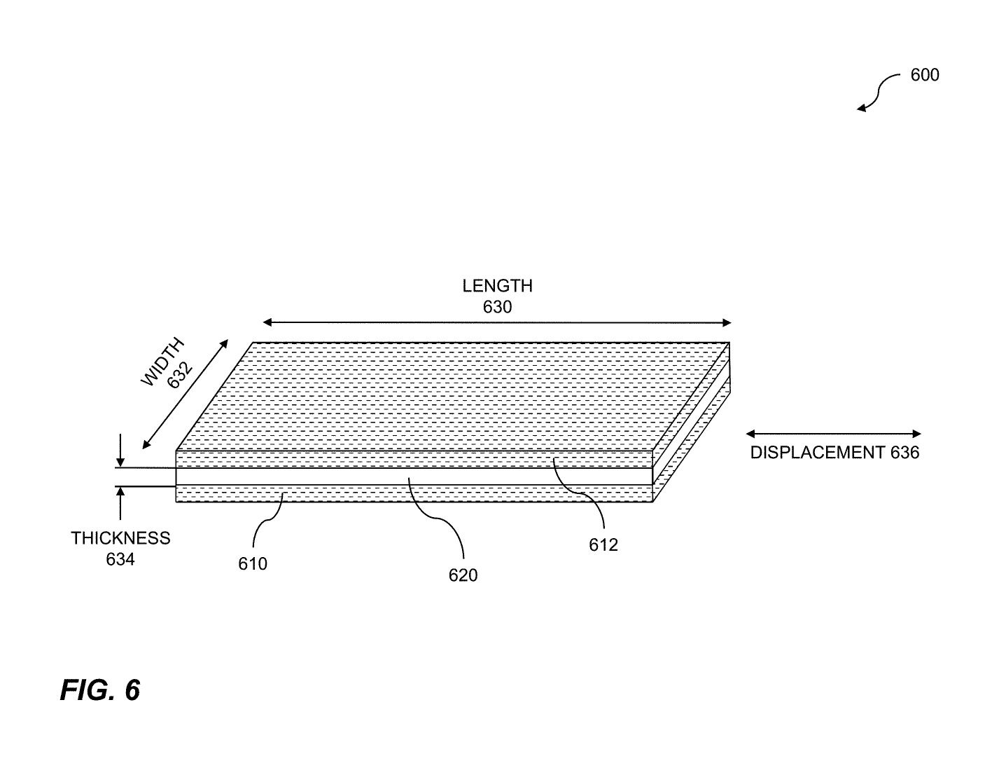

FIG. 6 illustrates detail of a capacitive sensor. Illustration 600 shows a three-dimensional view of a capacitive sensor implementation. The capacitive sensor has a length 630, a width 632. Embedded between conductive layers 610 and 612 is a dielectric material 620 with thickness 634. The conductive layers 610 and 612 can be attached to a fabric (not shown). The fabric may be a tape such as a therapeutic kinesiology tape, among other such tapes. Therapeutic kinesiology tape often exhibits properties of readily allowing deformation or stretching along only one axis. In this illustration, the length 630 deforms easily, but the width 632 does not readily deform. As the sensor is deformed or stretched along the length 630, a displacement 636 is indicated. However, it is clear that the aforesaid stretching will affect the dielectric material 620 and cause it to become thinner. This is due to the fact that, when one dimension of a three-dimensional solid material with finite volume is expanded, another dimension must contract to maintain the constant, finite volume. The thinning of dielectric material 620 will result in increased capacitance across the conductive layers 610 and 612. The capacitance may be approximated using the general parallel plate capacitor equation C=K*Eo*A/d, where Eₒ is the permittivity of free space (8.854×10⁻¹²), K is the dielectric constant of the material, A is the overlapping surface area of the plates, d is the distance between the plates, and C is capacitance.

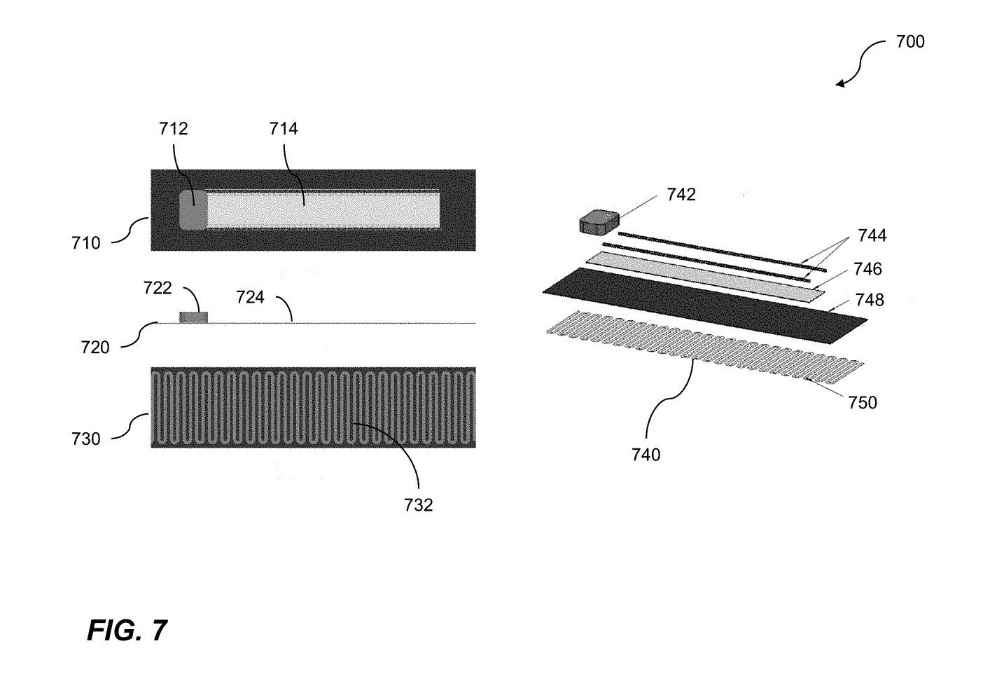

FIG. 7 illustrates fabric/sensor detail. Analysis, including body part deformation analysis, can be based on wearable body sensors placed on various body parts. Data is collected from a body sensor, where the body sensor is coupled to a fabric attached to a body part. Data in the form of electrical information can be collected from the body sensor. In embodiments, the fabric of the body sensor can be tape, where the tape can be physical therapy tape, therapeutic kinesiology tape, and so on. In other embodiments, the fabric of the body sensor can be a garment such as a cuff, a sock, a hat, a shirt, a pair of pants, etc. The electrical information can include capacitance, resistance, impedance, and/or inductance. The data from the body part is analyzed to determine the deformation of the body part. The deformation of the body part can be used to measure characteristics about the body part, to determine treatment of the body part, and so on.

A fabric/sensor detail is illustrated 700. As discussed, the fabric can include a tape, a garment, and so on. In embodiments, the fabric includes a woven material. A body sensor can be coupled to the fabric using a variety of techniques such as applying the sensor to the fabric, embedding the sensor in the fabric, etc. In embodiments, the woven material of the fabric can include an embroidered body sensor. In other embodiments, the woven material can include a woven body sensor. The woven material can include a Jacquard woven material in which the pattern of the Jacquard woven material includes the body sensor, and so on. Various views of fabric/sensor detail include a front view 710, a side view 720, and a bottom view 730. While a rectangular sensor shape is shown, other sensor shapes can include square, round, triangular, and so on.

The fabric/sensor detail includes a side layer view 740. The side layer view of the body sensor can include an electronics module 742. The electronics module is also shown on front view 710 as electronics module 712. The electronics module is also shown on side view 720 as the electronics module 722. The electronics module can be used to generate any signals required to operate the body sensors. The electronics module can be used to collect data from the body sensor. The side layer view can include stitching 744, where the stitching can be used for coupling the electronics module 742 to the sensor 746. The sensor 746 can be used to sense electrical information such as capacitance, resistance, impedance, inductance, and so on. The sensor is also shown on front view 710 as sensor 714 and on side view 720 as sensor 724. The side layer view can include a base layer 748, where the base layer can be used to support the sensor and other components, to stabilize the sensor and other components, etc. The side layer view can include an adhesive layer 750. The adhesive layer is also shown on bottom view 730 as adhesive layer 732. The adhesive layer can be used to attach one or more body sensors to a fabric, where the fabric can include tape, a garment, etc.

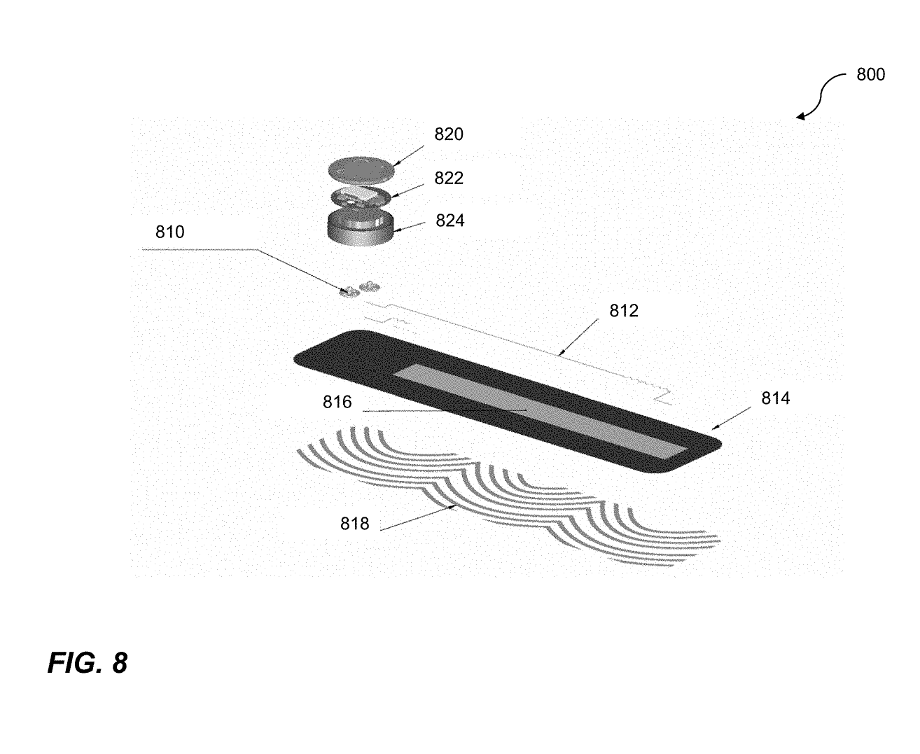

FIG. 8 illustrates fabric/sensor detail according to a first implementation. Illustration 800 includes an electronics module viewed in three pieces, base 824, circuitry 822, and cap 820. The base 824, circuitry 822, and cap 820 can be assembled together to provide an electronics module. The electronics module can be used to generate any signals required to operate the body sensors. The electronics module can be used to collect data from the body sensor. The electronics module base 824 can provide electronic coupling to the sensor 816 through the snap 810 and the stitching 812. The sensor 816 can be attached to a base layer 814. Base layer 814 can include an adhesive layer 818. The adhesive layer 818 can be used to attach one or more body sensors to a fabric, where the fabric can include tape, a garment, etc.

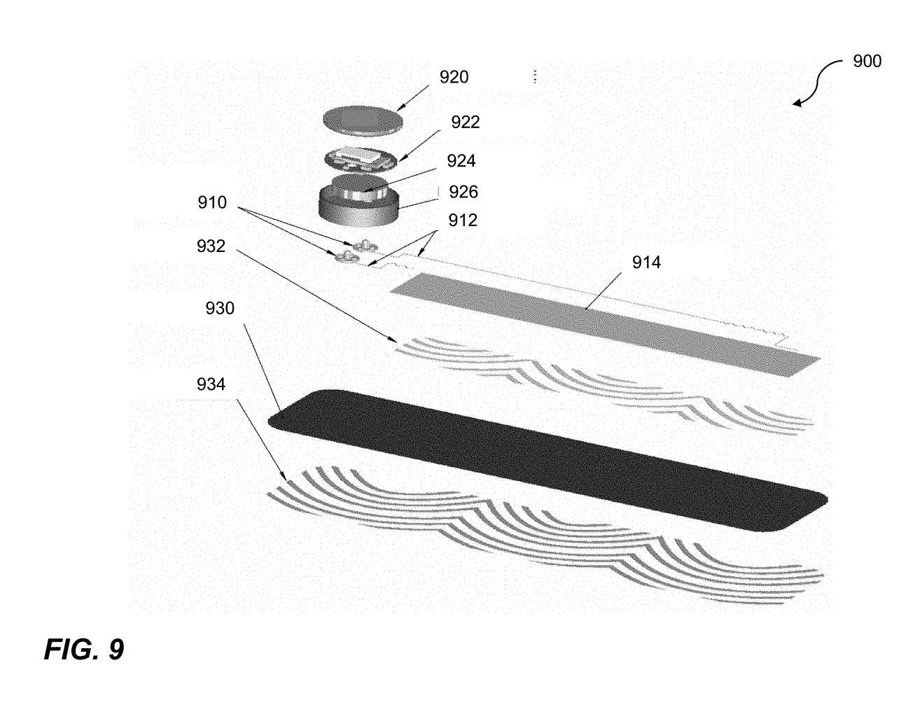

FIG. 9 illustrates fabric/sensor detail according to a second implementation. Illustration 900 includes an electronics module viewed in four pieces, base 926, battery 924, circuitry 922, and cap 920. The base 926, battery 924, circuitry 922, and cap 920 can be assembled together to provide an electronics module. The electronics module can be used to generate any signals required to operate the body sensors. The electronics module can be used to collect data from the body sensor. The electronics module base 926 can provide electronic coupling to the sensor 914 through snaps 910 and stitching 912. The stitching 912 can include discrete wires, woven wires, printed wires, and so on. The sensor 914 can be attached to a base layer 930 using adhesive layer 932. Base layer 930 can include an additional adhesive layer 934. The additional adhesive layer 934 can be used to attach one or more body sensors to a fabric, where the fabric can include tape, a garment, etc.

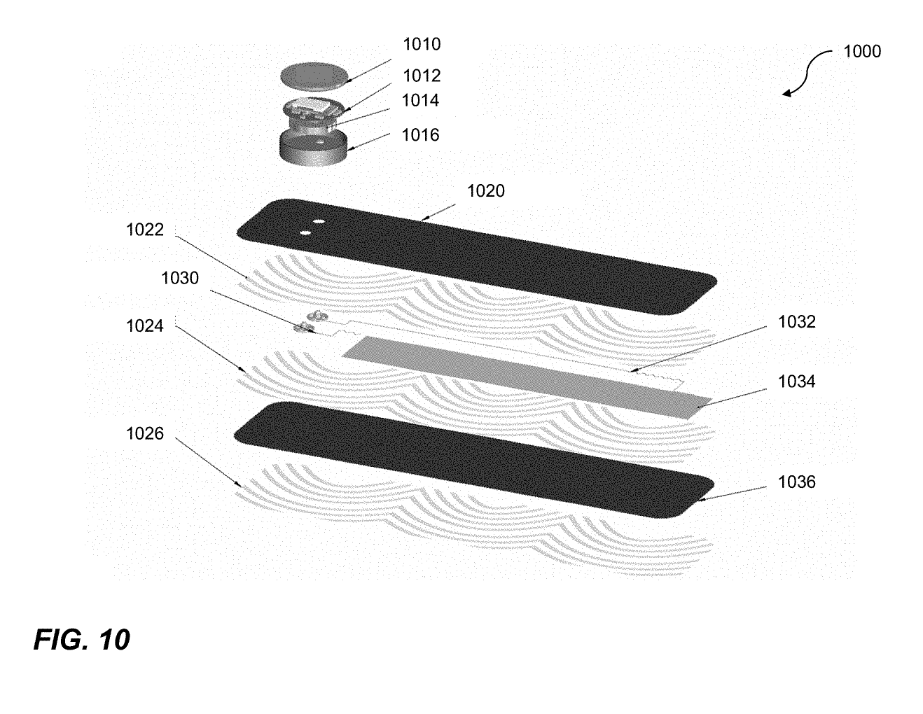

FIG. 10 illustrates fabric/sensor detail according to a third implementation. Illustration 1000 includes an electronics module viewed in four pieces, base 1016, battery 1014, circuitry 1012, and cap 1010. The base 1016, battery 1014, circuitry 1012, and cap 1010 can be assembled together to provide an electronics module. The electronics module can be used to generate any signals required to operate the body sensors. The electronics module can be used to collect data from the body sensor. The electronics module base 1016 can provide electronic coupling to a sensor 1034 through a protective layer 1020 using a snap 1030 and stitching 1032. The stitching 1032 can include discrete wires, woven wires, printed wires, and so on. The sensor 1034 can be attached to a protective layer 1020 with an adhesive layer 1022. The sensor 1034 can be attached to a base layer 1036 using another adhesive layer 1024. Base layer 1036 can include yet another adhesive layer 1026. This third adhesive layer 1026 can be used to attach one or more body sensors to a fabric, where the fabric can include tape, a garment, etc.

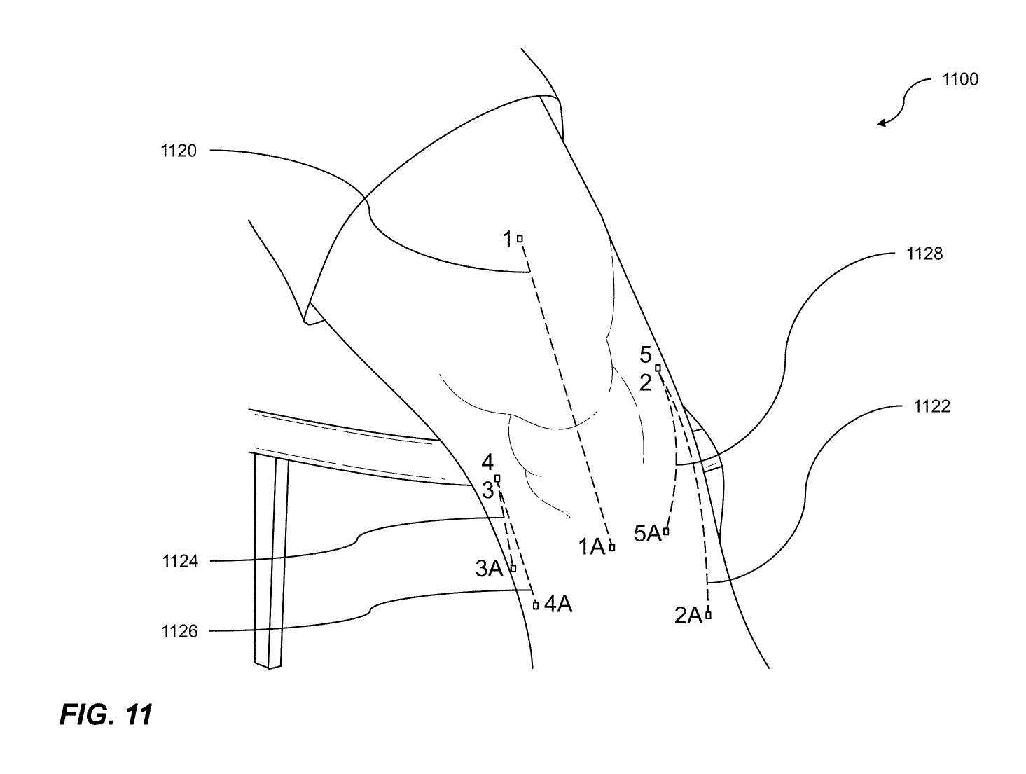

FIG. 11 shows anchor points for sensor placement on a leg. Body part deformation analysis using wearable body sensors includes collecting data from a body sensor.

The body sensor is coupled to a fabric attached to a body part, and the body sensor provides electrical information based on a deformation of the body part. Anchor points for sensor placement are shown in illustration 1100. The anchor points can include 1 to 1A 1120, 2 to 2A 1122, 3 to 3A 1124, 4 to 4A 1126, 5 to 5A 1128, and so on. Other anchor points for sensor placement can also be used. Similar anchor points can be used for sensor placement for other body parts such as the neck, back shoulders, elbows, wrists, hips, ankles, and so on. The anchor points for sensor placement can be chosen to sense deformations of specific body parts. For example, with respect to a knee, the anchor points for sensor placement can be chosen to determine deformation of a patellar tendon (PT), an anterior cruciate ligament (ACL), an anterolateral ligament (ALL), a lateral collateral ligament (LCL), a medial collateral ligament (MCL), and so on. Based on the determined deformation of the body part, body part treatment can be based on the analyzing data from the body part. The treatment of the body part can include one or more of medical techniques, physical therapy, occupational therapy, athletic training, strengthening, flexibility, endurance, conditioning, or rehabilitation therapy treatment.

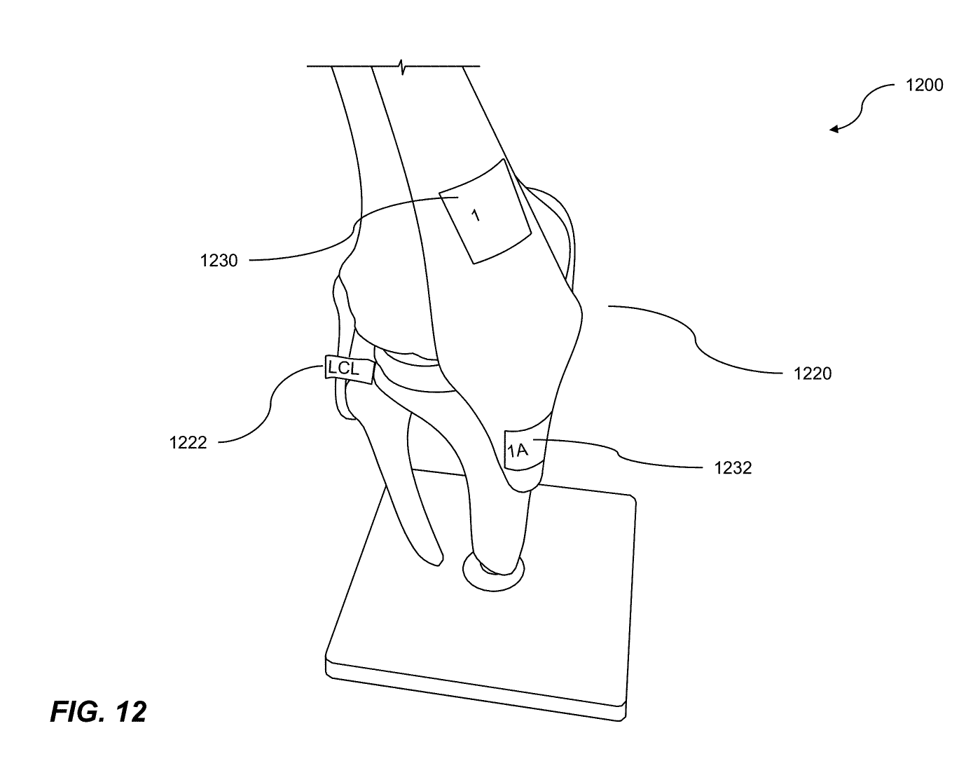

FIG. 12 illustrates anchor point anatomical detail. Body part deformation analysis is based on wearable body sensors. Data is collected from a body sensor. The body sensor is coupled to a fabric attached to a body part, and the body sensor provides electrical information based on a deformation of the body part. The data from the body sensor is analyzed to determine the deformation of the body part. Body part treatment can be based on the analysis of the deformation of the body part. Body part treatment can include one or more of medical techniques, physical therapy, occupational therapy, athletic training, strengthening, flexibility, endurance, conditioning, or rehabilitation therapy treatment. Anchor point anatomical detail is illustrated 1200. Anchor points 1 1230 and 1A 1232 can form a line along tendon 1220, along which one or more body sensors can be aligned. Other alignments can also be used. In embodiments, a body sensor can include two or more body sensors. The two or more body sensors can be aligned along line 1-1A, or aligned along another line (not shown). The two or more body sensors can be aligned based on other anchor points or multiple anchor points, as described elsewhere. The alignment of the one or more body sensors can be used to determine a deformation of the body part, such as the knee shown. The deformation can be part of analyzing a particular element of a body part or body part joint, such as ACL 1222. The alignment of the one or more sensors can be used for other analyzing. In embodiments, the analyzing can include performing a symmetry evaluation. The symmetry evaluation can include an evaluation of a similar body part such as comparing a left knee to a right knee, a left wrist to a right wrist, a left ankle to a right ankle, and so on. The symmetry evaluation can include an evaluation of a symmetrical operation for the body part, such as rotating up and down, rotating left and right, etc. The symmetry evaluation can include comparison to a “healthy” or normally functioning body part.

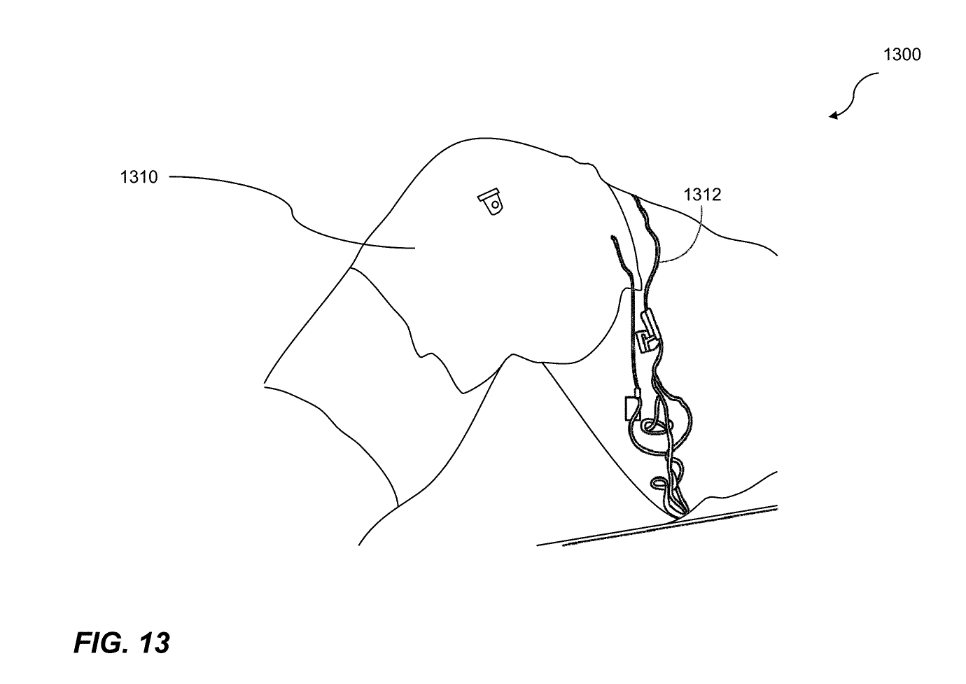

FIG. 13 shows right knee sensor attachment. Wearable body sensors are used for body part deformation analysis. One or more body sensors are coupled to a fabric attached to a body part. In embodiments, the body sensor can include two or more body sensors. The fabric to which the body sensor is coupled can be tape such as physical therapy tape and therapeutic kinesiology tape, a garment such as a glove, hat, sock, shirt, and pants, etc. Electrical information data such as resistance, capacitance, impedance, and/or inductance can be collected from the body sensor. The data from the body part is analyzed to determine the deformation of the body part. The deformation of the body part can be used to measure characteristics of the body part, to determine treatment for the body part, and so on. Right knee sensor attachment 1300 is shown. One or more body sensors coupled to fabric such as tape 1310 can be attached to the body part (knee). The attachment of the right knee sensor coupled to fabric such as tape 1310 can be based on anchor points for the alignment of the sensors, as described elsewhere. The electrical information that can be based on the deformation of the body port can be collected using a wired 1312 technique. In other embodiments, the electrical information can be collected using wireless techniques such as Wi-Fi, Bluetooth, near field communication (NFC), ZigBee, infrared (IF), and so on.

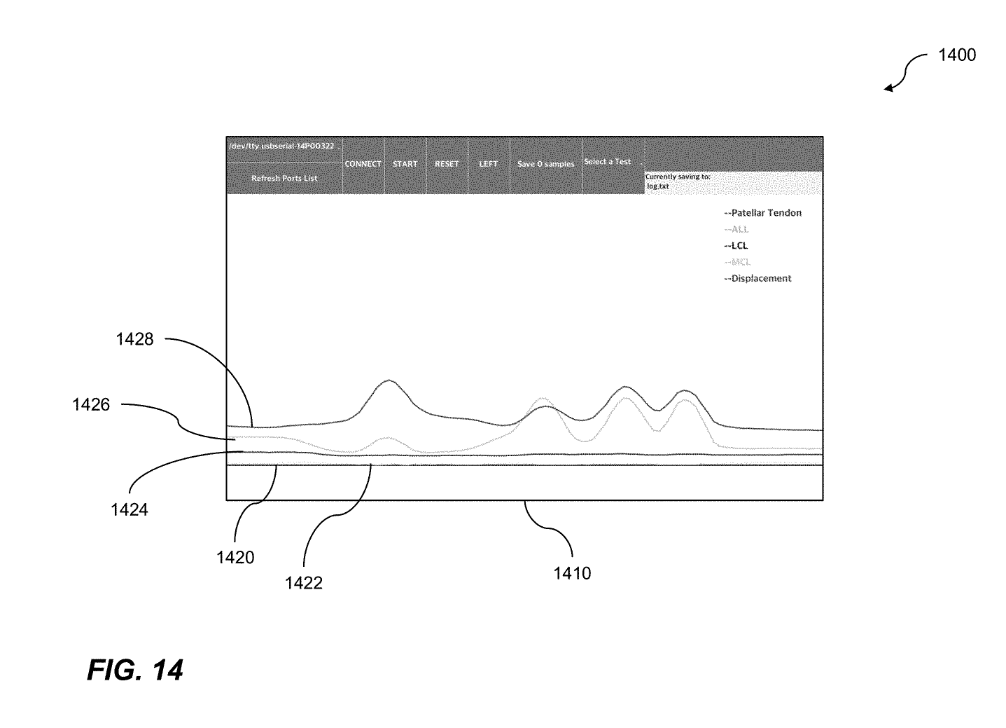

FIG. 14 illustrates electrical information from sensors. Deformation analysis can be based on data collected from wearable body sensors coupled to various body parts. A body sensor is coupled to a fabric attached to a body part. Electrical information data can be collected from the body sensor. The fabric of the body sensor can be tape such as physical therapy tape and therapeutic kinesiology tape, a garment, and so on. The electrical information can include resistance, capacitance, impedance, and/or inductance. The data from the body part is analyzed to determine the deformation of the body part. The deformation of the body part can be used to measure characteristics about the body part, to determine treatment of the body part, and so on. Electrical information data that can be gathered from sensors coupled to a fabric attached to a body part can be plotted and analyzed, compared to electrical information data from a “control” or healthy body part, and so on. Illustration 1400 shows electrical information data collected from multiple body part sensors and displayed in graphical format 1410. The electrical sensor information data can include signal 1424 from a patellar tendon (PT), signal 1422 from an anterolateral ligament (ALL), signal 1420 from a lateral collateral ligament (LCL), signal 1426 from a medial collateral ligament (MCL), and so on. The electrical information data collected from the sensors can also include displacement data 1428. The electrical information data can be augmented with physiological data, additional data collected from an inertial measurement unit (IMU), etc.

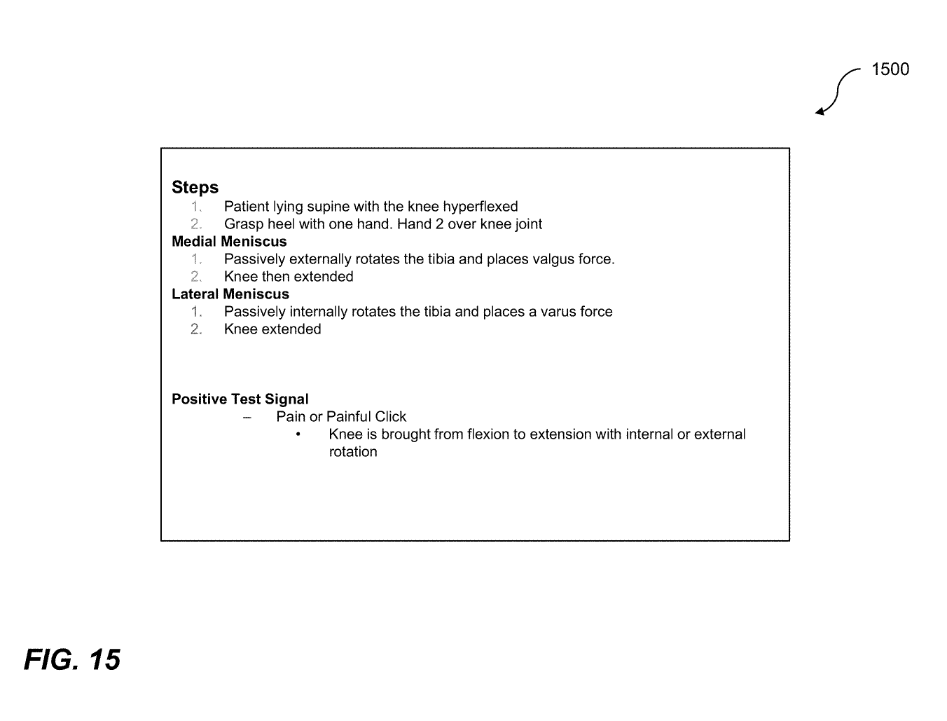

FIG. 15 provides steps for knee evaluation testing. The knee evaluation testing results can be automatically captured and analyzed using deformation analysis. The deformation analysis can be based on data collected from wearable body sensors coupled to various body parts. A body sensor is coupled to a fabric attached to a body part, in this case, a knee. Electrical information data can be collected from the body sensor. The fabric of the body sensor can be tape such as physical therapy tape and therapeutic kinesiology tape, a garment, and so on. The electrical information can include resistance, capacitance, impedance, and/or inductance. The data from the body part is analyzed to determine the deformation of the knee. The deformation of the knee can be used to measure characteristics about the knee, to determine treatment of the knee, to compare a healthy knee with an injured knee, and so on. Knee evaluation testing 1500 can include the steps of: (1) having a patient lying supine with the knee under evaluation hyperflexed, and (2) grasping the heel of the leg under evaluation with one hand while the other hand is placed over the knee joint. For evaluation of medial meniscus integrity, for example, the steps can further include: (3) passively rotating the tibia with the hand on the heel and placing a valgus force on the knee with the other hand, and (4) repeating step 3 with the knee under evaluation extended. Many other such tests can be included with deformation data being gathered by the one or more body part sensors. Objective data can be captured that is quantifiable well beyond typical reporting techniques. Based on the analysis of the data, treatment for the body part can be proposed. The treatment can include one or more of medical, physical therapy, occupational therapy, athletic training, strengthening, flexibility, endurance, conditioning, or rehabilitation therapy treatment.

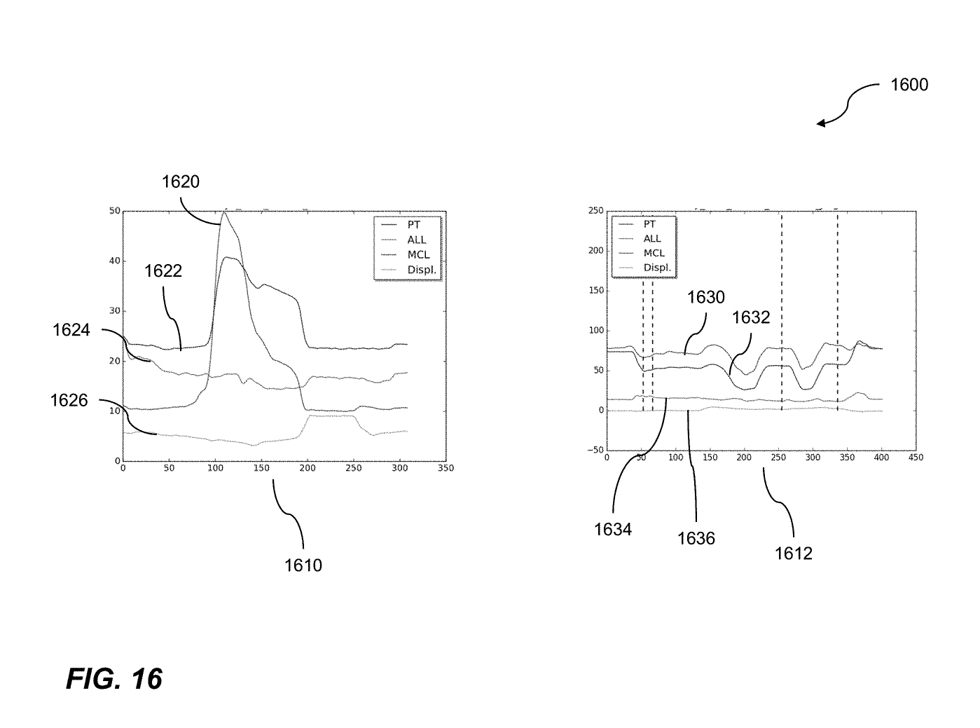

FIG. 16 illustrates deformation analysis. Wearable body sensors can be used for body part deformation analysis. Data is collected from a body sensor, where the body sensor is coupled to a fabric attached to a body part, and the body sensor provides electrical information based on a deformation of the body part. The fabric can be tape, where the tape can be physical therapy tape, therapeutic kinesiology tape, and so on. The electrical information can include resistance, capacitance, impedance, and/or inductance. The data from the body part is analyzed to determine the deformation of the body part. The deformation of the body part can be used to determine treatment of the body part.

Deformation analysis of a knee 1600 is shown. Deformation analysis can include deformation of one or more body parts including a knee, shoulder, elbow, wrist, hand, finger, thumb, ankle, foot, toe, hip, torso, spine, arm, leg, neck, jaw, head, back, and so on. Deformation analysis of a knee 1600 can be based on a McMurray circumduction test. The deformation analysis can include a lateral meniscus tests, a medial meniscus test, and so on. The test results can include deformation data relating to a patellar tendon (PT), an anterior cruciate ligament (ACL), a lateral collateral ligament (LCL), a medial collateral ligament (MCL), an anterolateral ligament (ALL), etc. A healthy right knee 1612 can be compared to another knee 1610. The healthy right knee 1612 and the other knee 1610 can be compared to determine the impact of an injured knee. A healthy right knee 1612 can show MCL deformation 1630, while the other knee 1610 can show MCL deformation 1620. Similarly, healthy right knee PT deformation 1632 can be compared to anatomical knee deformation 1622. Healthy right knee ALL deformation 1634 can be compared to anatomical knee deformation 1624. And healthy right knee displacement 1636 can be compared to anatomical knee displacement 1626. Based on the deformation analysis for the injured knee, treatment can be planned for the injured knee. Treatment can include one or more of medical, physical therapy, occupational therapy, athletic training, strengthening, flexibility, endurance, conditioning, or rehabilitation therapy treatment, and so on.

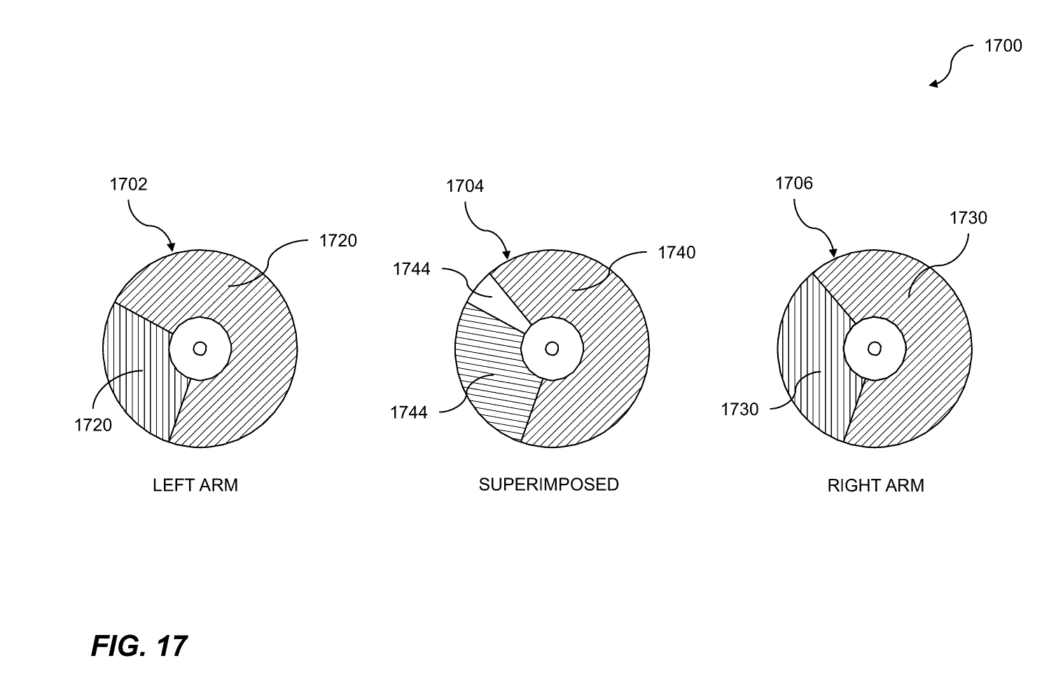

FIG. 17 illustrates deformation analysis of symmetric body parts. Symmetric body parts can be pairs of anatomically similar structures which normally function similarly. Symmetric body parts can include arms, legs, elbows, knees, ankles, hip components, right and left fingers, and so on. Often a patient normally has similar functionality for symmetric body parts. However, if an injury occurs, or other such abnormal functioning occurs, the injured one of the symmetric body parts can exhibit different deformation characteristics. In many cases, the differences are very difficult to detect through visual means, and even through computer-assisted visual means. Therefore accurate body part deformation data can be critical to determine the extent of an injury and the proper treatment plan thereof.

Illustration 1700 shows a comparison 1704 of left arm functionality 1702 and right arm functionality 1706. The functionality can be represented as a donut graph showing an extended deformation measurement and a flexed deformation measurement. Left arm functionality 1702 shows an extension deformation represented by the size of a donut arc 1720, and a flexion deformation represented by the size of another donut arc 1722. Similarly, right arm functionality 1706 shows an extension deformation represented by the size of a donut arc 1730, and a flexion deformation represented by the size of yet another donut arc 1732. Comparison 1704 shows a superposition of the deformation data for the left arm functionality 1702 and the right arm functionality 1706. Arcs 1740 and 1742 of superposition donut comparison 1704 represent the deformation donut arcs 1720 and 1722 of the left arm, when superimposed on right arm deformation donut arcs 1730 and 1732, respectively, and the resulting arc 1744 represents a deformation delta between the right and left arms. Arc 1744 clearly shows a readily-apparent difference between the functionality of the left and right arms. For example, a patient can be evaluated for arm, shoulder, neck, and back functionality by collecting deformation data from wearable body sensors while the patient performs a pull-up. The patient may exhibit subtle difference in the position of his left shoulder in comparison to his right shoulder at the height of the pull-up (chin over the bar at its highest position). The difference can be an indication of an injury or other functional impairment that can be treated based on an analysis of the deformation data collected on symmetric body parts.

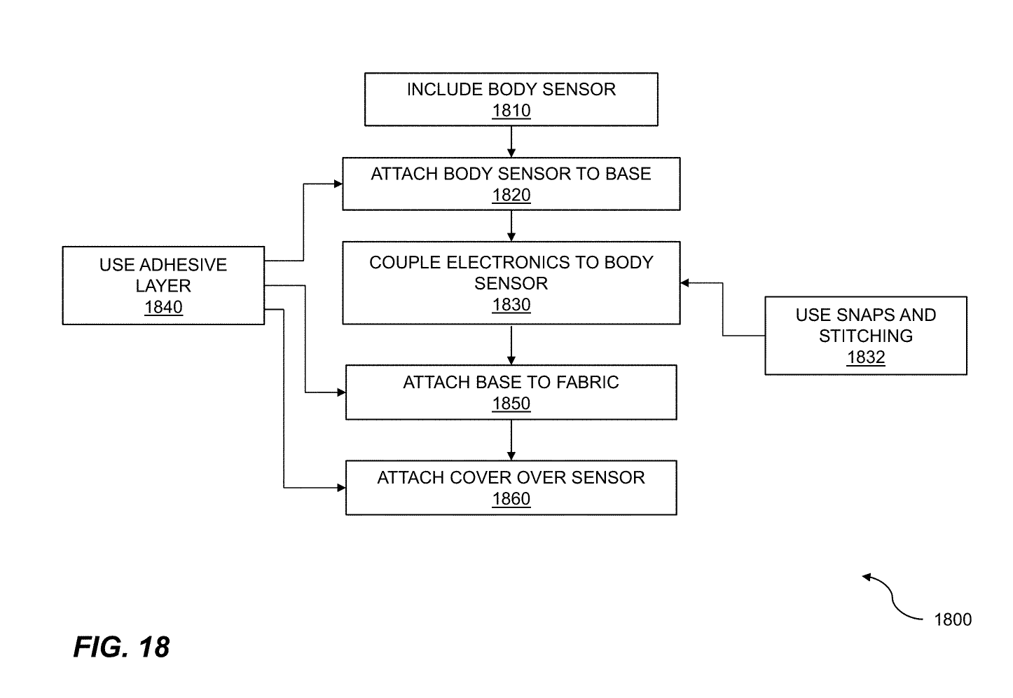

FIG. 18 is a flow diagram for body part sensor assembly. The flow 1800 includes including a body part sensor 1810. The body part sensor 1810 that is included can be a wearable body sensor. Wearable body sensors can be used for body part deformation analysis. One or more body sensors can be attached to a fabric attached to a body part. In embodiments, the body sensor can include two or more body sensors. The fabric to which the body sensor is coupled can be tape such as physical therapy tape and therapeutic kinesiology tape, a garment such as a glove, hat, sock, shirt, pants, etc. Electrical information data such as resistance, capacitance, impedance, and/or inductance can be collected from the body sensor. The data from the body part can be analyzed to determine the deformation of the body part. The deformation of the body part can be used to measure characteristics about the body part, to determine treatment for the body part, and so on.

The flow 1800 includes attaching a body sensor to a base 1820. The base can be used for the stability and careful handling of a delicate body sensor. The attaching the body sensor to a base 1820 can use an adhesive layer 1840. The flow 1800 includes coupling electronics to the body sensor 1830. The electronics can be used to generate any signals required to operate the body sensors. The electronics can be used to collect data from the body sensor. The electronics can use snaps and stitching 1832 to provide the coupling. The snaps can provide easy attachment and detachment of the electronics. The stitching can provide electrical connection and can comprise discrete wires, embedded wires, printed wires, and the like. The flow 1800 can include providing attaching the base to a fabric 1850. The fabric to which the body sensor is attached can be tape such as physical therapy tape and therapeutic kinesiology tape, a garment such as a glove, hat, sock, shirt, pants, etc. The attaching the base to a fabric 1850 can use an adhesive layer 1840. The flow 1800 can include attaching a cover over the sensor 1860. The cover can provide additional stability, handling protection, and use protection for a delicate sensor. The attaching a cover over the sensor 1860 can use an adhesive layer 1840.

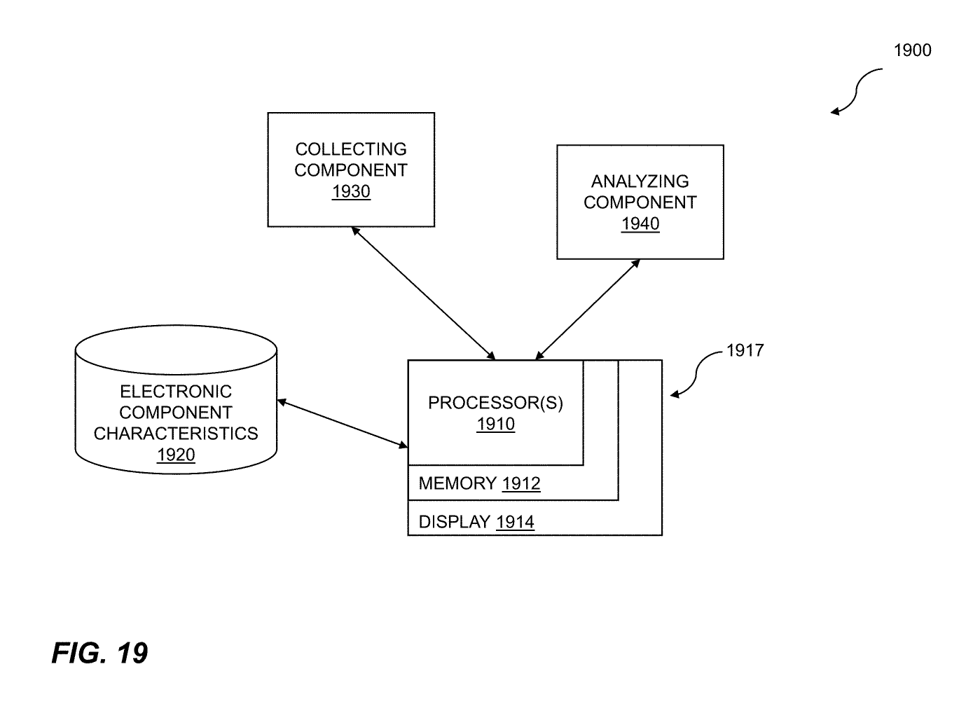

FIG. 19 is a system for body part analysis. Wearable body sensors can be used to analyze body part deformation. The wearable body sensors can include an embroidered body sensor, a woven body sensor, and so on. The wearable body sensors can include tape such as physical therapy tape, therapeutic kinesiology tape, etc. The body sensor can be applied to a body portion, worn on the body portion, encompass a body portion, etc. The wearable sensors can be used for collecting data from the body portion. The body sensor can be coupled to a fabric attached to a body portion, and the body sensor can provide electrical information such as capacitance, resistance, impedance, etc. The fabric can include a garment such as a sock, hat, shirt, pants, belt, and so on. The electrical information can be based on a deformation of the body part. Data collected from the body sensor can be analyzed to determine the deformation of the body part. The data relating to the deformation of the body part can be used for body part treatment including medical techniques, physical therapy, occupational therapy, athletic training, strengthening, flexibility, endurance, conditioning, or rehabilitation therapy treatment.

The system 1900 can include a collecting component 1930, an analyzing component 1940, an electronic component characteristics module 1920, and an analysis computer 1917. The analysis computer 1917 can comprise one or more processors 1910, a memory 1912 coupled to the one or more processors 1910, and an optional display 1914 configured and disposed to present user interface information. The display functionality can be integrated into the analysis computer 1917 or included in a separate device, such as a smartphone, tablet, laptop computer, or other external device capable of displaying data. The electronic component characteristics module 1920 can include a database and/or lookup table including empirically derived values, and can also include calibration data. The analyzing component 1940 can comprise one or more processors, a battery coupled to the one or more processors, a communication device, and so on. The collecting component 1930 can include resistance and/or capacitance measuring hardware and can include hardware for measuring current, voltage, resistance, capacitance, impedance, and/or inductance. A generating component (not shown) can include hardware for generating direct current and/or alternating current signals used for obtaining resistance and/or capacitance measurements. Typically, the current values are low (e.g. microamperes) and in embodiments, the frequency range includes signals from about 100 hertz to about 1 megahertz.

The system 1900 can include an apparatus for body part analysis comprising: a body sensor coupled to a fabric, wherein the fabric is attachable to a body part and wherein the body sensor provides electrical information based on a deformation of the body part; and a processor coupled to the body sensor, wherein the processor analyzes the data from the body sensor to determine the deformation of the body part. The system 1900 can include a computer program product embodied in a non-transitory computer readable medium for body part analysis, the computer program product comprising code which causes one or more processors to perform operations of: collecting data from a body sensor, wherein the body sensor is coupled to a fabric attached to a body part and the body sensor provides electrical information based on a deformation of the body part; and analyzing the data from the body sensor to determine the deformation of the body part. The system 1900 can include a computer system for body part analysis comprising: a memory which stores instructions; one or more processors attached to the memory wherein the one or more processors, when executing the instructions which are stored, are configured to: collect data from a body sensor, wherein the body sensor is coupled to a fabric attached to a body part; and the body sensor provides electrical information based on a deformation of the body part; and analyze the data from the body sensor to determine the deformation of the body part.

Each of the above methods may be executed on one or more processors on one or more computer systems. Embodiments may include various forms of distributed computing, client/server computing, and cloud based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A processor-implemented method for body part analysis comprising:
collecting data from a body sensor, wherein:
the body sensor is coupled to a fabric attached to a body part; and
the body sensor provides electrical information based on a deformation of the body part; and
analyzing the data from the body sensor to determine the deformation of the body part.

2. The method of claim 1 wherein the body part comprises a joint and the deformation includes bending of the joint.

3. The method of claim 2 wherein the electrical information provides information on an angle through which the joint bends.

4. The method of claim 1 wherein the fabric attached to the body part comprises tape.

5-6. (canceled)

7. The method of claim 1 wherein the collecting further comprises collecting data from a second body sensor.

8-9. (canceled)

10. The method of claim 7 wherein the second body sensor provides information on deformation of a joint beyond an angle of deformation for the joint.

11. The method of claim 10 wherein the second body sensor provides information on rotation of the joint.

12. The method of claim 1 further comprising determining anchor points for the body part, wherein the anchor points enable placement of the body sensor coupled to the fabric.

13. The method of claim 12 further comprising securing the fabric to the anchor points.

14. (canceled)

15. The method of claim 1 wherein the analyzing includes performing a symmetry evaluation.

16-19. (canceled)

20. The method of claim 1 wherein the deformation includes an angular motion.

21. The method of claim 20 further comprising determining an angle for the body part based on the deformation.

22. The method of claim 1 wherein the analyzing is performed to compare preoperative deformation with postoperative deformation.

23. The method of claim 1 wherein the analyzing is performed to compare deformation over time as part of physical therapy.

24. The method of claim 1 wherein the fabric comprises a brace.

25. (canceled)

26. The method of claim 1 wherein the fabric comprises a garment.

27-31. (canceled)

32. The method of claim 1 wherein the deformation comprises an anatomical evaluation.

33. The method of claim 1 wherein the deformation comprises a range of motion of the body part.

34. The method of claim 1 wherein the deformation comprises a ligament laxity.

35. The method of claim 1 wherein the deformation comprises a stability of the body part.

36. The method of claim 35 wherein the stability is analyzed for one or more repetitions of motion by the body part.

37-40. (canceled)

41. The method of claim 1 further comprising augmenting the data with additional data collected from an inertial measurement unit.

42. (canceled)

43. The method of claim 41 wherein the data and the additional data are used in a virtual reality representation of a body associated with the body part.

44. The method of claim 1 further comprising body part treatment, wherein the body part treatment is based on the analyzing.

45-47. (canceled)

48. The method of claim 1 wherein the data includes rotation of a joint, flex parameters of the joint, and varus and valgus tendencies of the joint, and further comprising collecting additional data from a second body sensor, wherein the additional data from the second body sensor provides information on deformation of the joint beyond an angle of deformation for the joint and rotation of the joint.

49. (canceled)

50. A computer program product embodied in a non-transitory computer readable medium for body part analysis, the computer program product comprising code which causes one or more processors to perform operations of:
collecting data from a body sensor, wherein:
the body sensor is coupled to a fabric attached to a body part; and
the body sensor provides electrical information based on a deformation of
the body part; and
analyzing the data from the body sensor to determine the deformation of the body part.

51. A computer system for body part analysis comprising:
a memory which stores instructions;
one or more processors attached to the memory wherein the one or more processors, when executing the instructions which are stored, are configured to:
collect data from a body sensor, wherein:
the body sensor is coupled to a fabric attached to a body part; and
the body sensor provides electrical information based on a deformation of the body part; and
analyze the data from the body sensor to determine the deformation of the body part.
